(19) United States
(12) Patent Application Publication
(10) Pub. No.: US 2007/0038089 A1
(43) Pub. Date: Feb. 15, 2007

(54) Transurethral diagnostic method and treatment method using ultrasonic endoscope

(75) Inventors: Tadashi Hatano, Tokyo (JP); Shoji Koga, Tokyo (JP); Koji Shimomura, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., TOKYO (JP)

(21) Appl. No.: 11/477,671

(22) Filed: Jun. 29, 2006

Related U.S. Application Data
(60) Provisional application No. 60/695,231, filed on Jun. 29, 2005.

(51) Int. Cl. A61B 8/00

(52) U.S. Cl. 600/437

(57) ABSTRACT

An ultrasonic endoscope has an ultrasonic probe, an observation optical system and a channel for inserting a medical instrument. A transurethral diagnostic method using the ultrasonic endoscope comprising step of inserting an insertion portion of the ultrasonic endoscope toward a target diagnostic portion through a urethral, step of starting transmission of an ultrasonic wave from the ultrasonic probe which has reached the vicinity of the target diagnostic portion and displaying at least a B-mode image of the target diagnostic portion on a screen, step of identifying a puncture position from the B-mode image displayed on the screen, and step of puncturing the puncture position identified on the screen with a medical instrument through the channel for inserting the medical instrument.

### BACKGROUND OF THE INVENTION

1. Field of the Invention

The present invention relates to a transurethral diagnostic method and a treatment method by inserting an insertion portion of an ultrasonic endoscope from a urethra to the vicinity of a target portion and then, by guiding a medical instrument through a channel for inserting the medical instrument provided at the ultrasonic endoscope for carrying out biopsy for a diagnosis or treatment.

2. Description of the Related Art

A diagnosis of a suspicious prostate carcinoma has been made by digital rectal palpation, prostatic specific antigen or transrectal ultrasonic inspection. However, it is necessary to sample a tissue of a prostate gland and to inspect the tissue by a microscope to make a definite diagnosis of a prostate carcinoma.

When sampling a tissue of a prostate gland, a prostate carcinoma biopsy method is known that an ultrasonic probe 110 is inserted from a rectum 100 and directed toward a prostate gland 101 as shown in FIG. 1 under observation of ultrasonic observation and a biopsy needle 111 is inserted so as to sample the tissue. And if it is determined to be a prostate carcinoma at this tissue sampling, radical prostatectomy for total extirpation of the prostate gland is performed in general.

With regard to an enlarged prostate gland, a treatment method or the like is known that an endoscope, not shown, is inserted into a urethra 102 and a medical instrument for laser radiation or an RF probe is inserted through a channel for inserting the medical instrument provided at the endoscope to necrotize a tissue. Note that reference numeral 103 in the figure denotes a bladder.

### SUMMARY OF THE INVENTION

A transurethral diagnostic method using an ultrasonic endoscope having an ultrasonic probe, an observation optical system and a channel for inserting a medical instrument comprises a step of inserting an insertion portion of the ultrasonic endoscope toward a target diagnostic portion through the urethra, a step of starting transmission of an ultrasonic wave from the ultrasonic probe which has reached the vicinity of the target diagnostic portion and displaying at least a B-mode image of the target diagnostic portion on a screen, a step of identifying a puncture position from the B-mode image displayed on the screen and a step of puncturing the puncture position identified on the screen by a medical instrument through the channel for inserting the medical instrument.

The above and other objects, features and advantages of the invention will become more clearly understood from the following description referring to the accompanying drawings.

### BRIEF DESCRIPTION OF THE DRAWINGS

FIG. 1 is a view for explaining conventional transrectal tissue sampling while observing a prostate gland using an ultrasonic probe;

FIG. 2 is a view for explaining a construction example of an ultrasonic endoscope device;

FIG. 3 is a perspective view for explaining construction and relation of a tip-end rigid portion of the ultrasonic endoscope and an ultrasonic probe;

FIG. 4 is a view for explaining the construction of a puncture device, which is one of medical instruments;

FIG. 5 is a view for explaining the construction of the puncture device using a solid needle tube provided with a notch portion at a needle point;

FIG. 6 is a flowchart for explaining procedures of tissue sampling according to a transurethral diagnostic method performed using the ultrasonic endoscope device;

FIG. 7 is a view for explaining a state where an insertion portion of the ultrasonic endoscope is inserted into a urethra;

FIG. 8 is a view for explaining a B-mode image displayed on a screen of a display device;

FIG. 9 is a view for explaining a state where the needle tube is inserted into a prostate gland;

FIG. 10 is a sectional view of X-X line of FIG. 9 for explaining a state where an ultrasonic scanning range is rotated and changed;

FIG. 11 is a view for explaining a state where the needle tube is inserted into the prostate gland after the ultrasonic probe is moved toward a deep portion;

FIG. 12 is a view for explaining a state where the needle tube is inserted into the prostate gland after the ultrasonic probe is further moved toward the deep portion;

FIG. 13 is a view for explaining a state of the needle tube when a projecting angle is changed and the state of the needle tube when the projecting length is changed;

FIG. 14 is a view for explaining a state where a balloon provided at the ultrasonic endoscope inserted to the vicinity of the prostate gland is expanded;

FIG. 15 is a view for explaining a state where the expanded balloon is brought into close contact with the total inner-circumference of a urethral wall;

FIG. 16 is a flowchart for explaining procedures of a transurethral treatment method of an enlarged prostate gland using the ultrasonic endoscope device;

FIG. 17 is a view for explaining a state where a medicinal solution is injected through the needle tube projecting into the prostate gland;

FIG. 18 is a view for explaining a state where the medicinal solution is injected through the needle tube projected into the prostate gland after the ultrasonic probe has been moved toward the deep portion;

FIG. 19 is a view for explaining the B-mode image displayed on the screen of the display device;

FIG. 20 is a view for explaining a Doppler's image displayed on the screen of the display device;

FIG. 21 is a flowchart for explaining procedures of a transurethral treatment method of a prostate carcinoma performed using the ultrasonic endoscope device;

FIG. 22 is a view for explaining a work for finding a cancer portion in the prostate gland by twisting operation of the insertion portion;

FIG. 23 is a view for explaining the B-mode image including a cancer displayed on the screen of the display device;

FIG. 24 is a view for explaining a state where the medicinal solution is injected through the needle tube projected into the cancer portion of the prostate gland; and

FIG. 25 is a view for explaining a diagnosis and treatment performed by inserting the ultrasonic endoscope to the bladder through the urethra.

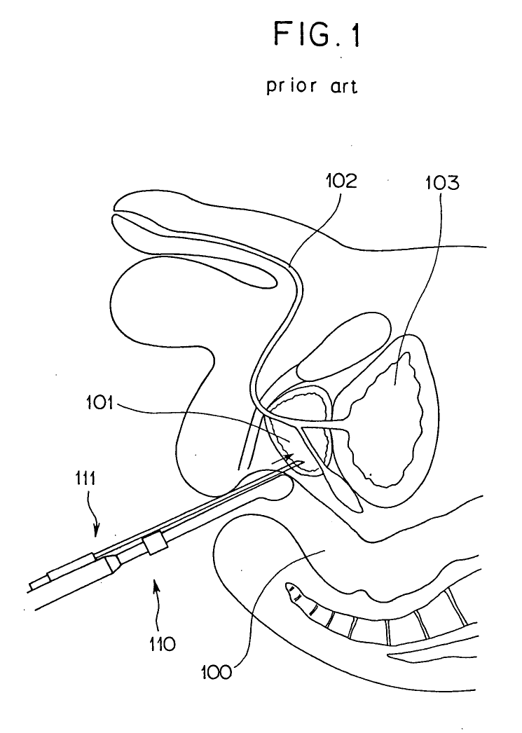

### DETAILED DESCRIPTION OF THE PREFERRED EMBODIMENTS

An embodiment of the present invention will be described below referring to the attached drawings.

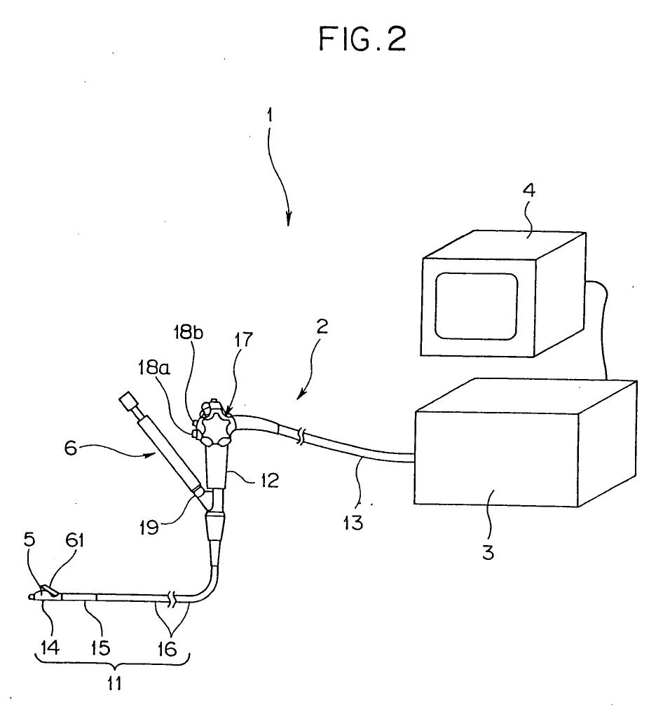

As shown in FIG. 2, an ultrasonic endoscope device 1 mainly comprises an ultrasonic endoscope 2, an ultrasonic observing device 3 and an observing monitor 4.

The ultrasonic endoscope 2 comprises an insertion portion 11, an operation portion 12 and a universal cord 13. An ultrasonic probe 5 is provided at a tip end side of the insertion portion 11. An endoscope connector (not shown) and an ultrasonic connector (not shown) to be connected to the ultrasonic observing device 3 are provided at a base end portion of the universal cord 13. The universal cord 13 is extended from a side portion of the operation portion 12.

The insertion portion 11 of this embodiment has flexibility, for example, and is elongated and inserted into the urethra, and the ultrasonic endoscope 2 is used for a transurethral diagnosis or treatment. The insertion portion 11 comprises a tip-end rigid portion 14, a curved portion 15 capable of being curved and a flexible pipe portion 16 which is small in diameter and lengthy and has flexibility in connection with each other. The operation portion 12 is disposed on a base end side of the insertion portion 11. At the operation portion 12, an angle knob 17 for curving the curved portion 15 in a desired direction, a water supply button 18a for water supply operation, a suction button 18b for suction operation and so on are provided. Moreover, a medical instrument insertion port 19 is provided on the tip end portion side of the operation portion 12. Through the medical instrument insertion port 19, a puncture device 6, which is a medical instrument for performing a diagnosis or treatment, an RF (Radio Frequency) probe (not shown) or the like is arranged. And a needle tube 61 of the puncture device 6 is projected from a tip-end opening, which will be described later, provided at the tip-end rigid portion 14.

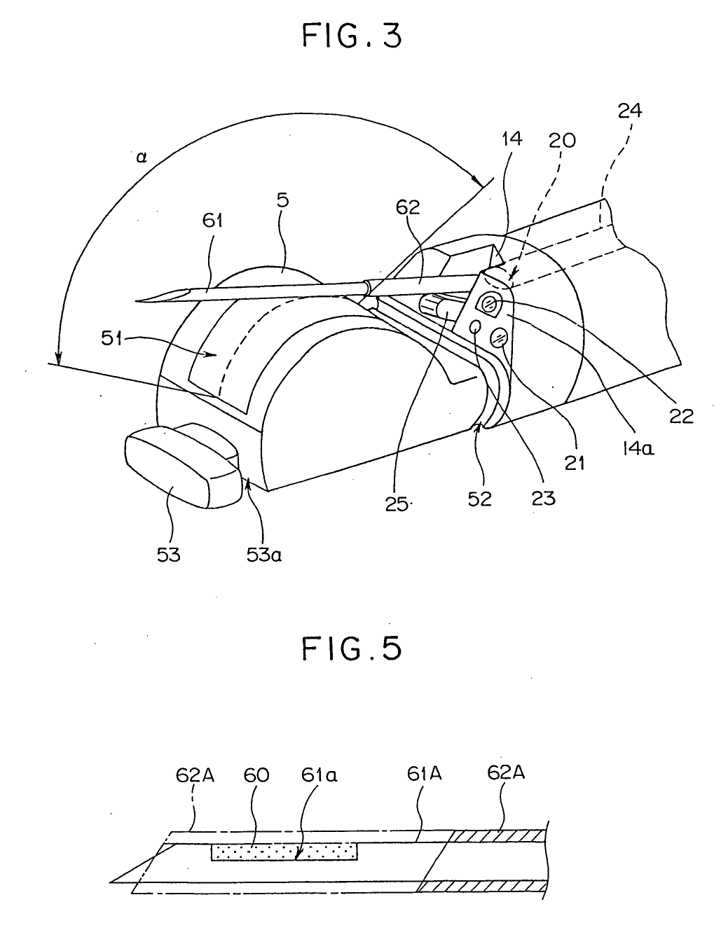

As shown in FIG. 3, at a tip-end inclined portion 14a of the tip-end rigid portion 14, a tip-end opening 20, an illumination lens cover 21 constituting an illumination optical system, an observation lens cover 22 constituting an observation optical system and a water supply nozzle 23 are provided.

The tip-end opening 20 and the medical instrument insertion port 19 communicate with each other through a channel for inserting a medical instrument (hereinafter referred to as insertion channel) 24. Therefore, by mounting the puncture device 6 at the medical instrument insertion port 19 and performing hand operation, for example, the needle tube 61 is projected from the tip-end opening 20.

A raising base 25 capable of swing is provided in the vicinity of the tip-end opening 20. The raising base 25 is swung/operated by a raising lever (not shown) provided at the operation portion 12. Specifically, in the state where the needle tube 61 is projected from the tip-end opening 20 as shown in the figure, the raising table 25 is swung by operating the raising lever. Then, with the swing of the raising table 25, the needle tube 61 is moved within a scanning range α in the figure as appropriate.

The ultrasonic probe 5 is provided at the tip end side of the tip-end rigid portion 14. The ultrasonic probe 5 is a convex type and comprises a plurality of piezoelectric elements (not shown) for sending/receiving an ultrasonic wave arranged. The ultrasonic probe 5 is parallel with the longitudinal direction of the insertion portion 11 and has an ultrasonic scanning surface 51 having a fan-shaped scanning range a shown by an arrow. The tip-end opening 20 is provided so that the needle tube 61, for example, projected from the tip-end opening 20 of the insertion channel 24 is included in the ultrasonic scanning range a.

Reference numeral 52 in the figure denotes a balloon attaching groove portion in which one end of a balloon is disposed. Reference numeral 53 denotes a projection portion for attaching a balloon and constitutes a groove portion 53a in which the other end of the balloon is disposed between it, and a tip end face of the ultrasonic probe 5. An injection port (not shown) is provided in the vicinity of the balloon attaching groove portion 52, and the balloon is expanded by supplying an ultrasonic transmission medium into the balloon through the injection port. And the expanded state of the balloon is controlled by an operator through an injection amount of the ultrasonic transmission medium into the balloon.

The ultrasonic probe 5 sends and receives an ultrasonic wave for diagnosis to obtain an ultrasonic image for diagnosis. A signal line is extended from the ultrasonic probe 5. The signal line extended from the ultrasonic probe 5 is inserted through the insertion portion 11, the operation portion 12 and the universal cord 13 and connected to a B-mode sending/receiving circuit (not shown) and a Doppler sending/receiving circuit (not shown) in the ultrasonic observing device 3. The B-mode sending/receiving circuit is connected to a B-mode image processing circuit (not shown) for generating a B-mode image based on a received signal. The Doppler sending/receiving circuit is connected to a Doppler signal processing circuit (not shown) for generating an image of blood flow information by detecting a Doppler component of the received signal. The B-mode image processing circuit and the Doppler signal processing circuit are connected to a digital scan converter (not shown) for converting the B-mode image and the Doppler image, which is an image on blood flow information, into a video signal.

On a screen 4a of a display device 4, the B-mode image and the Doppler image outputted from the ultrasonic observing device 3 are displayed in color, for example, separately or at the same time. In this embodiment, a light source portion (not shown) for supplying illuminating light to an illumination optical system of the ultrasonic endoscope 2 is provided in the ultrasonic observing device 3. The endoscope image is displayed on a screen of a display device, not shown, for example.

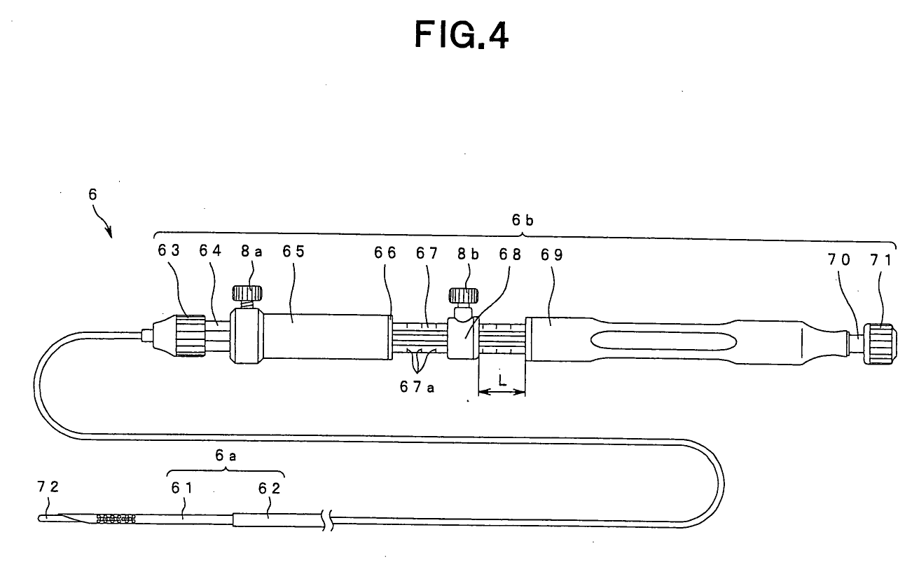

The puncture device 6 comprises, as shown in FIG. 4, a channel insertion portion 6a to be inserted into the insertion channel 24 and a handle portion 6b, which is an operation and grip portion provided on a base end side of this channel insertion portion 6a.

The channel insertion portion 6a mainly comprises a sheath 62 and the needle tube 61, and a stylet 72 is arranged capable of insertion/removal in the needle tube 61. The needle tube 61 is inserted and arranged in the sheath 62 capable of going forward/backward. The needle tube 61 is formed by a metal pipe such as a stainless pipe or a nickel titanium pipe, for example.

The handle portion 6b comprises a fixing ring 63, a sheath length adjusting member 64, a sheath length adjusting portion body 65, a sheath connection portion 66, an operation portion body 67, a stopper member 68, a slider 69, a suction base 70 and a stylet holding portion 71 arranged sequentially from the tip end side.

The fixing ring 63 is connected and fixed to a base portion provided at the medical instrument insertion port 19. The sheath length adjusting portion body 65 is a tubular member and slidably arranged on an outer circumferential surface of the sheath length adjusting member 64. The base end of the sheath 62 is integrally fixed to an inner hole of the sheath connection portion 66 by adhesion, for example. The base end of the needle tube 61 is integrally fixed to an inner hole of the suction base 70 by adhesion, for example. The stylet holding portion 71 is integrally provided at the base end of the stylet 72.

A first fixing screw 8a is provided at the sheath length adjusting portion body 65. By bringing the first fixing screw 8a into a loosened state, the sheath length adjusting portion body 65 is made slidable with respect to the sheath length adjusting member 64. And by tightening the first fixing screw 8a, the sheath length adjusting portion body 65 can be integrally fixed to the sheath length adjusting member 64 in the state where the sheath length adjusting portion body 65 is moved by a predetermined amount. By this, the length of the sheath 62 extending from the tip end of the fixing ring 63 constituting the handle portion 6b can be controlled.

A second fixing screw 8b is provided at the stopper member 68. By bringing the second fixing screw 8b into a loosened state, the stopper member 68 is made movable with respect to the operation portion body 67. And by tightening the second fixing screw 8b, the stopper member 68 is fixed to the operation portion body 67 in the state where the stopper member 68 is moved by a predetermined amount. A moved distance L of the stopper member 68 with respect to the slider 69 at this time is the traveling length of the slider 69, that is, the projecting length of the needle tube 61. It is to be noted that reference numeral 67a denotes a scale to be indication when setting an arrangement position of the stopper member 68. The scale 67a is provided on an outer circumferential surface of the operation portion body 67 with a predetermined interval.

The needle tube 61 is formed with its tip end portion in a sharp shape in FIGS. 3 and 4. This needle tube 61 has its tip end portion punctured into a target portion under observation of an ultrasonic image. After that, a syringe (not shown) or the like is connected to the suction base 70 to perform suction. By this, a tissue is taken into the needle tube 61. And the tissue taken into the needle tube 61 is recovered after the puncture device 6 is pulled out of the insertion channel 24. By this, sampling of the tissue is completed.

Also, as shown in FIG. 5, a puncture device 6A may be constituted using a solid needle tube 61A provided with a notch portion 61a at the needlepoint instead of the needle tube 61. In this puncture device 6A, an outer needle 62A in the shape of a pipe is provided in the sheath 62 (not shown in FIG. 5). The needle tube 61A is inserted into the outer needle 62A. A tip end of the outer needle 62A is also in a sharp shape. When sampling a tissue using the puncture device 6A, first, under observation of the ultrasonic image, the notch portion 61a of the needle tube 61A is made to reach a target portion. After that, the outer needle 62A shown by a solid line is advanced as shown by a chain line so as to completely cover an outside of the notch portion 61a. A tissue 60 at the target portion is cut off by the advancement of the outer needle 62A, and the tissue is taken into the notch portion 61a. And the tissue taken into the notch portion 61a of the needle tube 61A is recovered after the puncture device 6A is pulled out of the insertion channel 24. By this, sampling of the tissue is completed. The other constructions of the puncture device 6A is the same as those of the above puncture device 6, and illustration will be omitted.

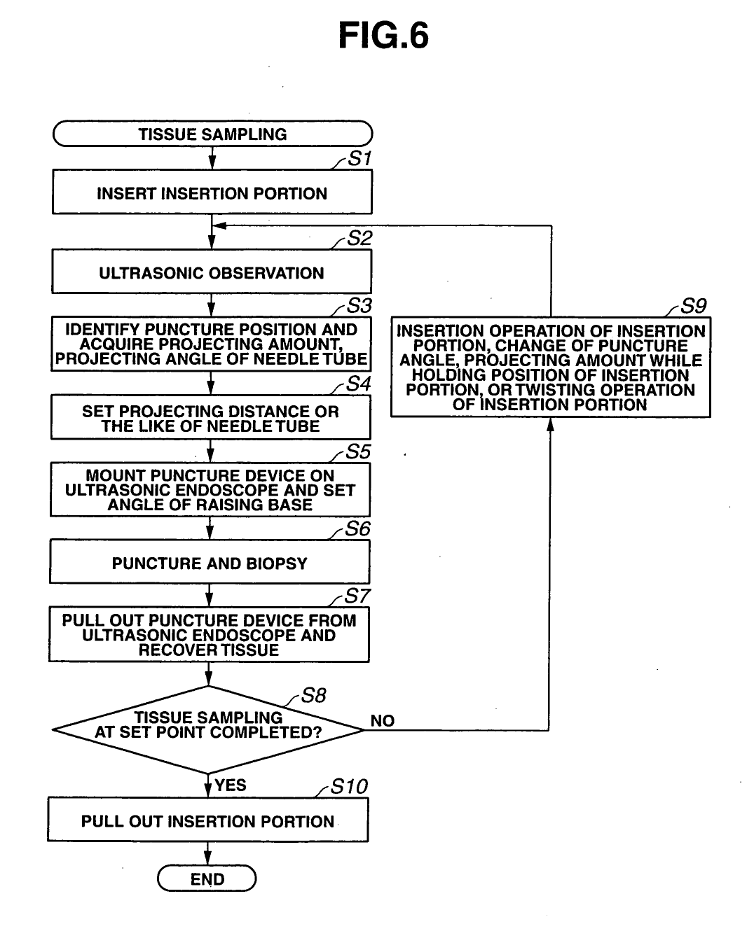

A transurethral diagnostic method using the ultrasonic endoscope device 1 will be described referring to FIGS. 6 to 13.

When making a transurethral diagnosis using the ultrasonic endoscope 2, an operator examines a pre-operation image such as a CT image and an MRI image as necessary so as to grasp the state of a prostate gland and determines a plurality of positions for tissue recovery in advance. And the ultrasonic endoscope device 1 is constructed by preparing a plurality of the puncture devices 6A as medical instruments for sampling a tissue of the prostate gland. After that, the operator samples the tissue of the prostate gland according to the procedures in FIG. 6 and then, determines if a patient has a prostate carcinoma or not.

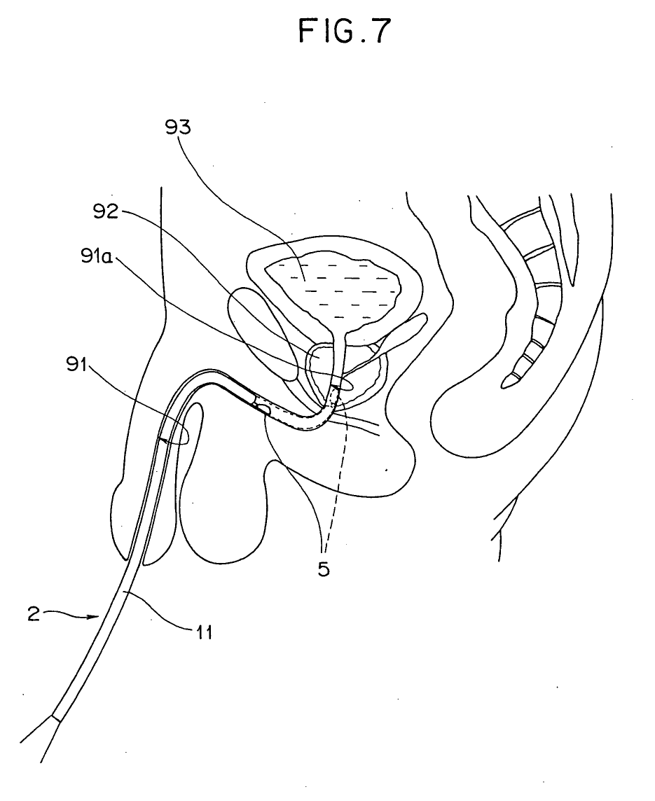

First, the operator inserts the insertion portion 11 of the ultrasonic endoscope 2 into a urethra 91 as shown in FIG. 7 as in Step S1. And while performing visual observation by an observing optical system provided at the ultrasonic endoscope 2 and ultrasonic observation using the ultrasonic probe 5 as appropriate, the operator inserts the ultrasonic probe 5 provided at the tip end of the insertion portion 11 toward the prostate gland 92, which is a target diagnostic portion. Reference numeral 93 denotes a bladder.

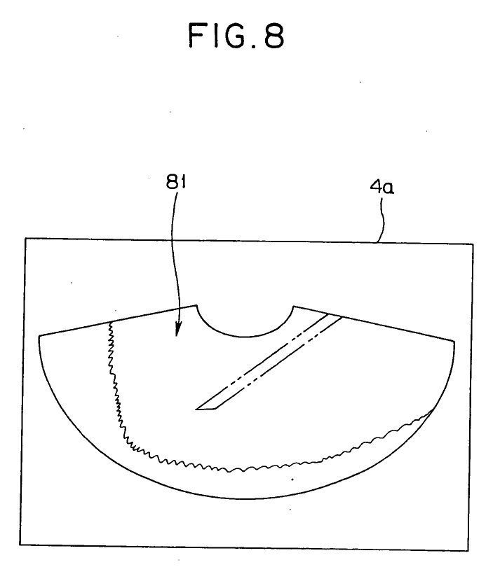

When the ultrasonic probe 5 reaches the vicinity of the prostate gland 92 as shown by a broken line in FIG. 7, the operator brings the ultrasonic probe 5 into close contact with a urethral wall 91a by curving operation or the like. And as shown in Step S2, the ultrasonic observation is started. Then, as shown in FIG. 8, a B-mode image of the prostate gland 92 is displayed on the screen 4a of the display device 4. Here, the operator observes a B-mode image 81 displayed on the screen 4a of the display device 4 and has a Doppler image displayed on the screen 4a in order to check a running state of a blood vessel as necessary and makes position control so as not to damage the blood vessel at puncture. And the operator observes the B-mode image 81 and identifies a puncture position as shown in Step S3 to make the position correspond to the tissue recovery position determined in advance. At this time, the operator acquires the projecting length of the needle tube 61 and the projecting angle, in other words, a tilting angle of the raising base 25 so that the needle tube 61 is brought into the puncture state as shown by a two-dot chain line.

Next, as shown in Step S4, by manipulating the first fixing screw 8a and the second fixing screw 8b of the puncture device 6A so as to control the length of the sheath 62 extending from the tip end of the fixing ring 63 and the projecting length of the needle tube 61, the routine goes on to Step S5. At Step S5, the operator has the needle tube 61 or the like accommodated in the sheath 62 and inserts the sheath 62 in that state into the insertion channel 24 from the medical instrument insertion port 19. After that, the fixing ring 63 of the puncture device 6A is connected and fixed to the base portion provided at the medical instrument insertion port 19. By this, the puncture device 6A is mounted at a predetermined position of the ultrasonic endoscope 2 in a predetermined state. After that, the operator adjusts the tilting angle of the raising base 25 to an angle acquired at Step S3.

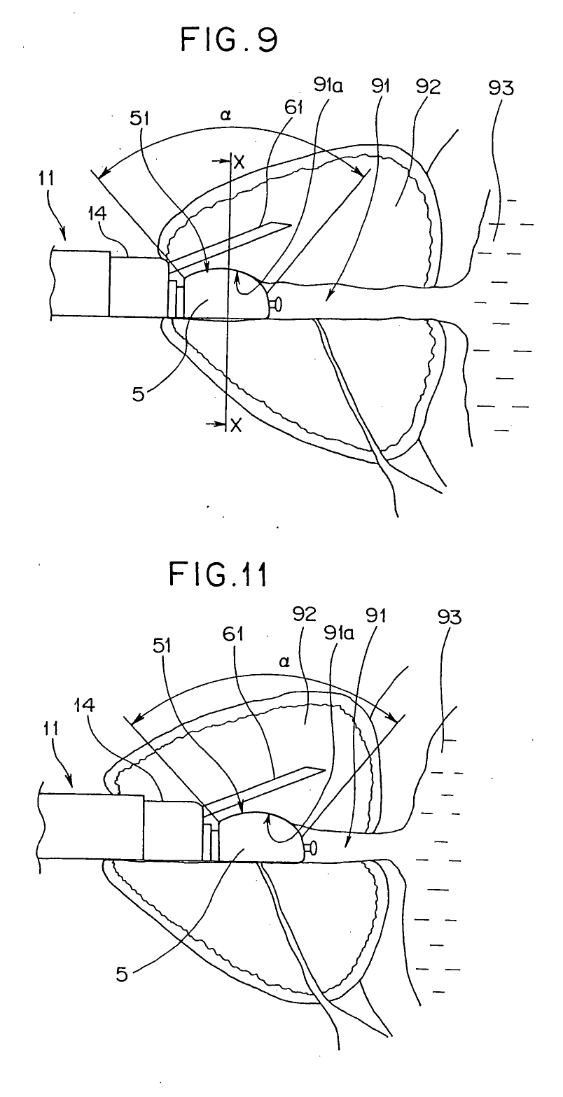

At Step S6, the operator performs puncture by the needle tube 61 toward the prostate gland 92 under ultrasonic guide. Then, as shown in FIG. 9, the needle tube 61 is inserted into the prostate gland 92. At this time, the operator checks a position of the tip end of the needle tube 61, puncture depth or the like on the B-mode image displayed on the screen 4a of the display device 4.

Next, the routine goes on to Step S7. At Step S7, the operator removes the fixing ring 63 having been connected and fixed to the base portion in order to recover the tissue and pulls out the puncture device 6A from the medical instrument insertion port 19. After that, the tissue is recovered. By this, tissue sampling at one location is finished. When the puncture device 6A is pulled out of the medical instrument insertion port 19, the operator moves on to Step S8. At Step S8, since the operator has not completed the tissue sampling at a preset location, the operator moves on to Step S9 in order to perform another tissue sampling. Here, the operator performs insertion operation of the insertion portion 11, change of projecting angle of the needle tube 61, change of projecting length of the needle tube 61 or twisting operation of the insertion portion 11 in order to carry out tissue sampling at a tissue recovery position determined before the operation.

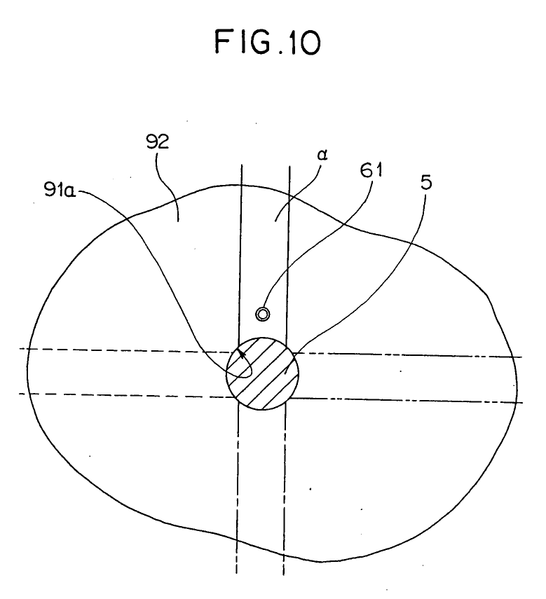

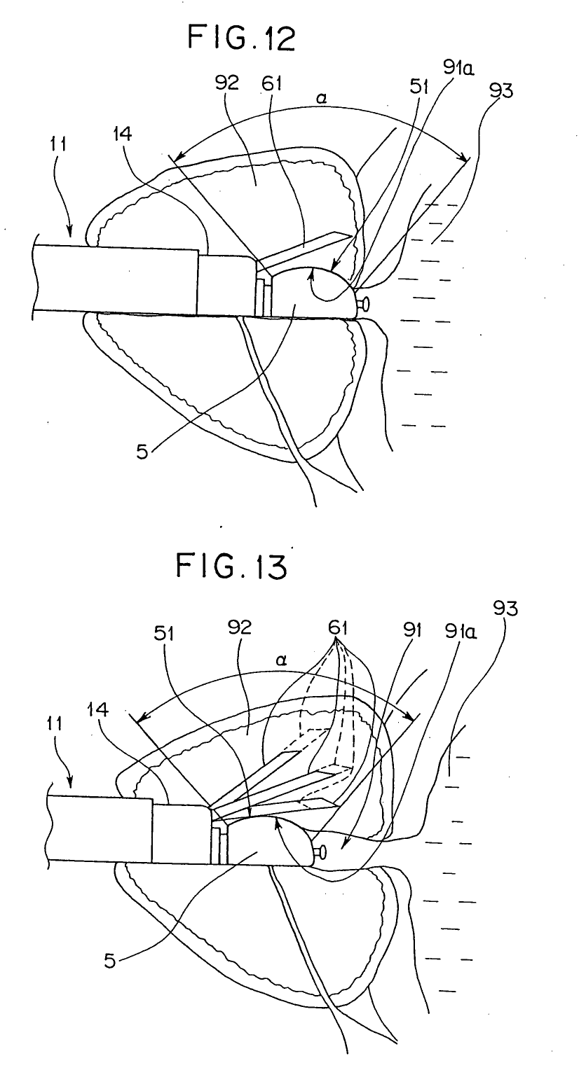

Specifically, by twisting operation of the insertion portion 11, the ultrasonic probe 5 is rotated with respect to the urethra 91 so that an ultrasonic scanning range a is changed as shown by a solid line, a broken line, a chain line and a two-dot chain line as shown in FIG. 10. And after the ultrasonic probe 5 is twisted and operated to a desired position, a tissue is sampled according to the procedures from Step S2 to Step S7. Also, by insertion operation of the insertion portion 11, the ultrasonic probe 5 is introduced toward a deep portion of the urethra 91 as shown in FIGS. 11 and 12. And after the ultrasonic probe 5 is introduced to the desired position, the tissue is sampled according to the procedures from Step S2 to Step S7. Moreover, by changing the tilting angle of the raising base 25, the position of the needle tube 61 is changed as shown by a solid line, for example, in FIG. 13, and by changing the projection length, the position of the tip end of the needle tube 61 is changed as shown by a broken line, for example, in the figure. The operator performs twisting operation, insertion operation, change of projecting angle and projecting length and repeats the procedures from Step S2 to Step S7 in order to sample the tissue.

And when the operator determines that sampling of the target tissue has been completed at Step S8, the routine goes on to Step S10, where the insertion portion 11 of the ultrasonic endoscope 2 is pulled out of the urethra 91 to finish the inspection. After that, the sampled tissue is given definite diagnosis pathologically.

In this way, the insertion portion of the ultrasonic endoscope is inserted into the urethra and guided to the vicinity of the prostate gland. After that, the needle tube of the puncture device is inserted to the prostate gland through the insertion channel provided at the ultrasonic endoscope under ultrasonic observation and transurethral tissue sampling of the prostate gland is performed visually. By this, the puncture position and puncture depth of the needle tube of the puncture device can be checked and direct puncture by the needle tube into the prostate gland can be carried out through the urethral wall. Therefore, tissue in the vicinity of a plurality of tissue recovery positions determined in advance upon examination of pre-surgery images such as CT images and MRI images can be sampled.

Also, as compared with the case of tissue sampling that a biopsy needle is inserted toward a prostate gland through a rectum, in tissue sampling of a prostate gland by transrectal ultrasonic inspection, spread of the sampled tissue to other portions can be prevented. This is caused by the fact that a surgery for a prostate carcinoma is total extirpation. That is, when a tissue is sampled in the transurethral manner, the portion where the sampled tissue spreads is limited to the urethra, and the urethra is extracted together with the entire prostate gland in the extirpation. Therefore, if the sampled tissue is a cancer cell, metastasis of the cancer cell to other portions can be prevented.

Moreover, as compared with the case of tissue sampling by inserting a biopsy needle toward a prostate gland through a rectum, in tissue sampling of a prostate gland by transrectal ultrasonic inspection, invasion can be reduced. That is, when puncture is performed in the transrectal manner, inflammation occurs at a portion punctured by the biopsy needle and adhesion is generated. And even if the sampled tissue is diagnosed as a cancer cell, it is difficult to do an operation till the inflammation subsides. And an operation with the adhesion existing heightens a fear of heavier bleeding and prolongs the operation time. On the other hand, waiting for the inflammation to subside would cause the cancer to progress during that period. In the meantime, in the tissue sampling of the prostate gland by transurethral ultrasonic inspection, even if the inflammation occurs, it is on the urethral wall portion to be extracted in the operation. Therefore, if the sampled tissue is diagnosed as a cancer cell, the prostate gland can be separated immediately after the diagnosis.

Also, in the case of transrectal tissue sampling by puncture by a biopsy needle toward a prostate gland through a rectum, location for tissue sampling is limited since tissue sampling by piercing a urethra can not be performed or the like. On the other hand, in the transurethral ultrasonic inspection, sampling location can be set over the whole region of the prostate gland. Therefore, by performing tissue sampling of a prostate gland over the whole region, a probability of discovering a prostate carcinoma is drastically improved.

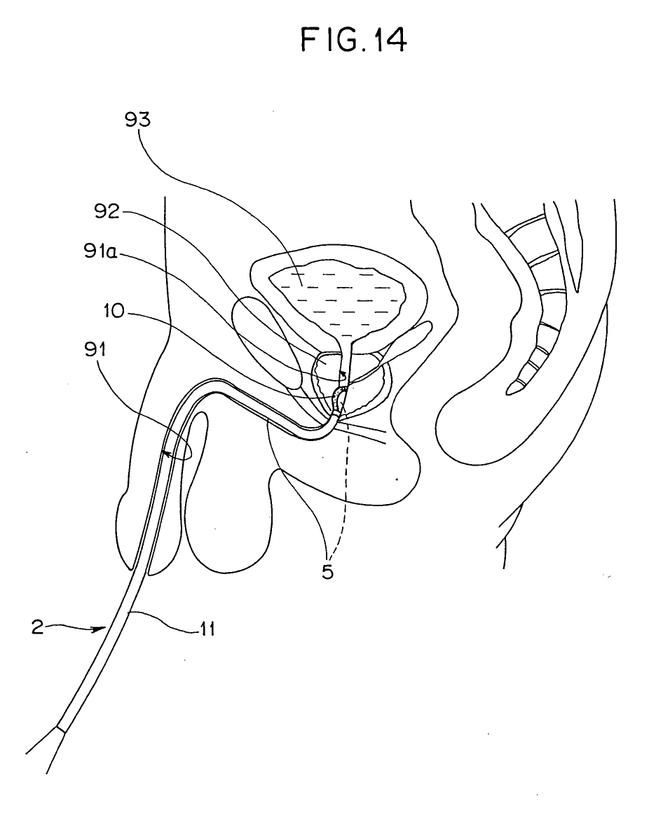

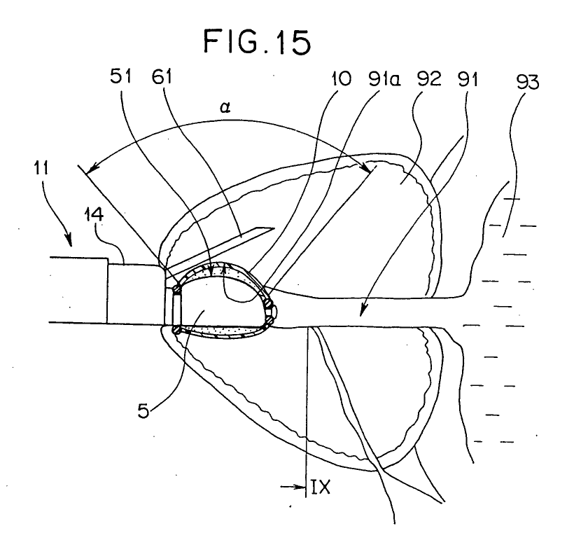

The ultrasonic probe 5 may be covered by a balloon 10 as shown in FIG. 14. By this, by expanding the balloon 10 in the middle of insertion in FIG. 14, for example, a gap is formed between the urethral wall 91a and the observation lens cover 22 so as to ensure a sufficient visual field of the endoscope. Also, by expanding/contracting the balloon 10 in the vicinity of a prostate gland as shown in FIG. 15, the balloon 10 is brought into close contact with the urethral wall 91a over the entire circumference, and the ultrasonic probe 5 can be arranged within the urethra 91 in a more stable state.

A transurethral treatment method of an enlarged prostate gland by the ultrasonic endoscope device 1 will be described referring to FIGS. 7, 8 and 16 to 20.

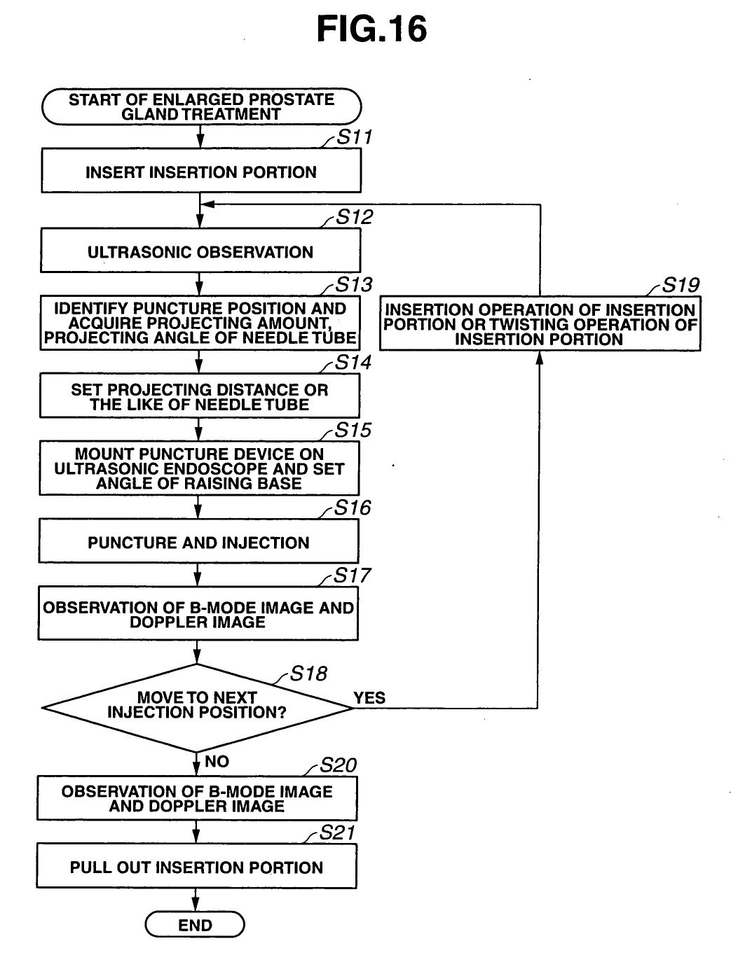

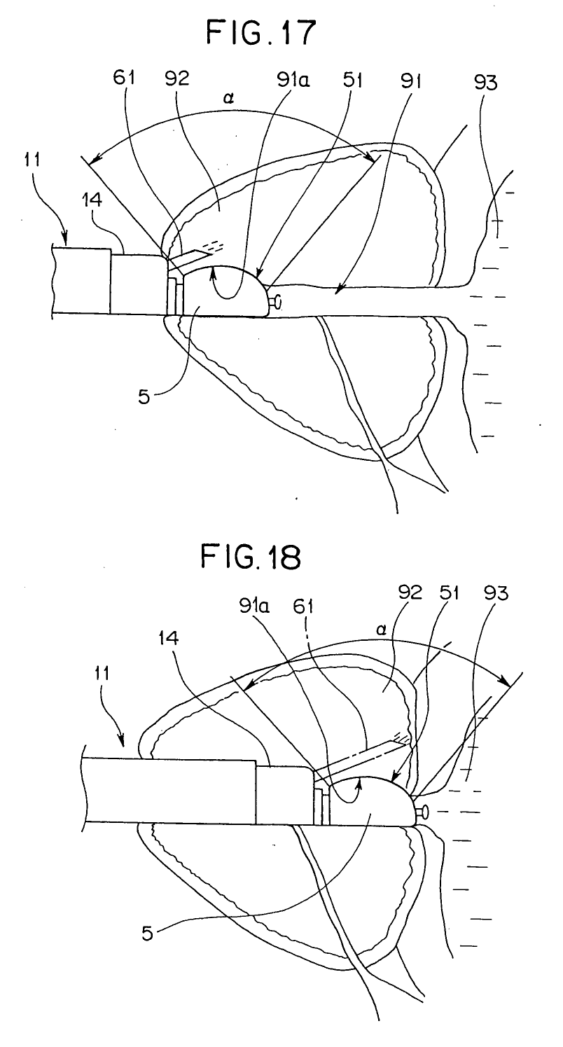

In the transurethral treatment for an enlarged prostate gland using the ultrasonic endoscope 2, an operator examines a pre-operation image such as a CT image and an MRI image as necessary so as to grasp the present state of the enlarged prostate gland and determines a plurality of positions for injecting medicinal solution or the like in advance. And the puncture device 6 for sampling a tissue of a prostate gland is prepared as a medical instrument, and a syringe for injecting ethanol or botulinus bacillus is prepared so as to constitute the ultrasonic endoscope device 1. After that, the operator proceeds with treatment for enlarged prostate gland according to procedures in FIG. 16. In this embodiment, ethanol is filled in the syringe.

First, as shown in Step S11, the operator inserts the insertion portion 11 of the ultrasonic endoscope 2 into the urethra 91 as shown in FIG. 7. And the ultrasonic probe 5 provided at the tip end of the insertion portion 11 is inserted toward the prostate gland 92, which is a target diagnostic portion, while performing visual observation by the observing optical system provided at the ultrasonic endoscope 2 and the ultrasonic observation by the ultrasonic probe 5.

When the ultrasonic probe 5 reaches the vicinity of the prostate gland 92 as shown in FIG. 7, the operator performs curving operation or the like to bring the ultrasonic probe 5 into close contact with the urethral wall 91a. And as shown in Step S 12, ultrasonic observation is started. Then, as shown in FIG. 8, the B-mode image of the prostate gland 92 is displayed on the screen 4a of the display device 4. Here, the operator observes the B-mode image 81 displayed on the screen 4a of the display device 4 and has a color Doppler image displayed on the screen 4a so as to check the running state of a blood vessel as necessary and to confirm the puncture position. And the operator identifies the puncture position as shown in Step S13 to make the position correspond to the injection position determined in advance. At this time, the operator acquires the projecting length and the projecting angle of the needle tube 61 so that the needle tube 61 is inserted as shown by a two-dot chain line.

Next, as shown in Step S14, by manipulating the first fixing screw 8a, the second fixing screw 8b of the puncture device 6, the length of the sheath 62 extending from the tip end of the fixing ring 63 and the projecting length of the needle tube 61 are controlled, and the routine goes on to Step S15. At Step S15, the operator inserts the sheath 62 accommodating the needle tube 61 or the like into the insertion channel 24 from the medical instrument insertion port 19. After that, the fixing ring 63 of the puncture device 6 is connected and fixed to the base portion provided at the medical instrument insertion port 19. By this, the puncture device 6 is mounted at a predetermined position of the ultrasonic endoscope 2 in a predetermined state. After that, the operator sets the tilting angle of the raising base 25 to an angle acquired at Step S13.

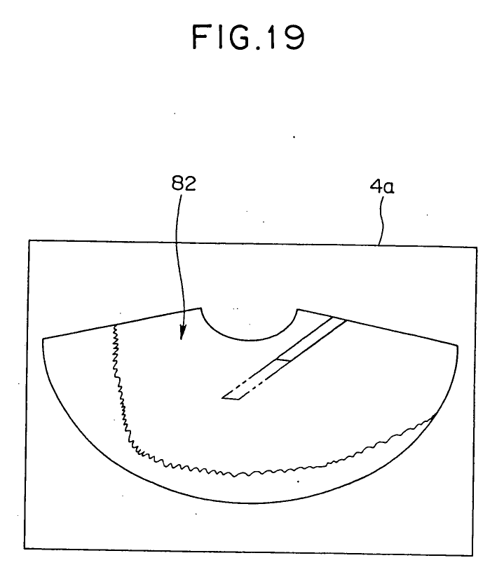

At Step S16, the operator performs puncture by the needle tube 61 toward the prostate gland 92 under ultrasonic guide. Then, as shown in FIGS. 17 and 18, the needle tube 61 is inserted into the prostate gland 92. At this time, images of the needle tube 61 projected as shown by a solid line in FIG. 17 and the needle tube 61 projected as shown by a chain line in FIG. 18 are displayed on the B-mode image 82 displayed on the screen 4a of the display device 4 as shown in FIG. 19. The operator checks a position of the tip end of the needle tube 61, puncture depth or the like on this B-mode image displayed on the screen 4a of the display device 4. After that, the syringe is attached to the suction base 70 and pushing-in operation is performed and a predetermined amount of ethanol is injected into the prostate gland under ultrasonic observation.

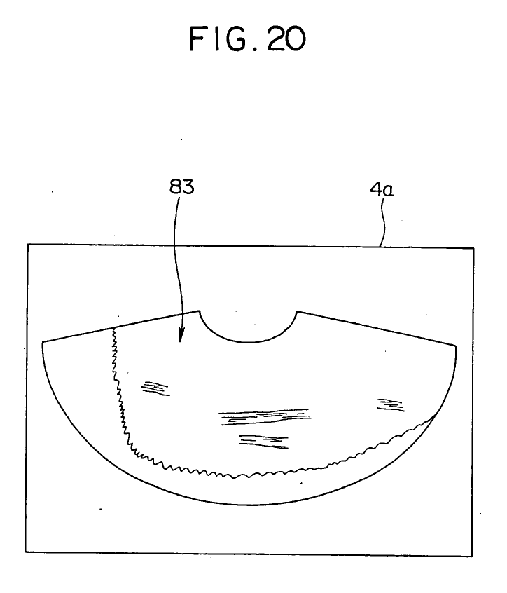

After ethanol injection, the operator accommodates the needle tube 61 projected into prostate gland in the insertion portion 11 as necessary, visually checks a blood flow state in a blood vessel by the color Doppler image 83 showing a blood flow shown in FIG. 20 as shown in Step S17, and can determine if there is a sign of occluding or not. After that, the routine goes on to Step S18. Here, when the operator determines that it is necessary to move to the next injection point, the routine goes on to Step S19. At Step S19, the operator performs twisting operation to twist the insertion portion 11, insertion operation to insert the insertion portion 11 into a deep portion or hand operation to change the projecting angle, projecting length. And a predetermined amount of ethanol is injected into the prostate gland according to the procedures from Step S12 to Step S16. And the blood flow state in the blood vessel can be visually checked as necessary by the color Doppler image 83 showing a blood flow shown in FIG. 20 as shown in Step S17. And at Step S18, when injection of the predetermined amount of ethanol into a present injection spot has been completed, the routine goes on to Step S20, where the blood flow state in the blood vessel can be also checked by the color Doppler image shown in FIG. 19. And after the operator has checked occluding on the color Doppler image, the routine goes on to Step S21, where the insertion portion 11 of the ultrasonic endoscope 2 is pulled out of the urethra 91 to finish the treatment.

In this way, the insertion portion of the ultrasonic endoscope is inserted into a urethra and guided to the vicinity of a prostate gland. After that, the needle tube of the puncture device is inserted into a desired position of the prostate gland under ultrasonic observation through the insertion channel provided at the ultrasonic endoscope, ethanol is injected visually and transurethral treatment is given to the enlarged prostate gland. By this, ethanol can be injected after checking a puncture position and puncture depth of the needle tube of the puncture device on the B-mode image, and the blood flow state in the blood vessel can be checked by checking the Doppler image after ethanol injection or the like so that effect of the injected ethanol can be determined in a short time.

In this embodiment, a treatment method to necrotize a tissue by injecting ethanol is shown, but instead of injecting ethanol into a prostate gland, botulinus bacillus with an action to reduce an enlarged prostate gland may be injected into a specific location of the enlarged prostate gland under ultrasonic observation.

This treatment using botulinus bacillus is described in U.S. Pat. No. 6,365,164, “USE OF NEUROTOXIN THERAPY FOR TREATMENT OF UROLOGIC AND RELATED DISORDERS”. The ethanol treatment is as described in “Transurethral ethanol injection therapy for prostatic hyperplasia; 3-year results”, Goya N, et al., Journal of Urology, September 2004; 172(3): 1017-20, PubMed, National Center for Biotechnology Information, National Library of Medicine”.

A transurethral treatment method for a prostate carcinoma using the ultrasonic endoscope device 1 will be described referring to FIGS. 7, 8 and 21 to 24.

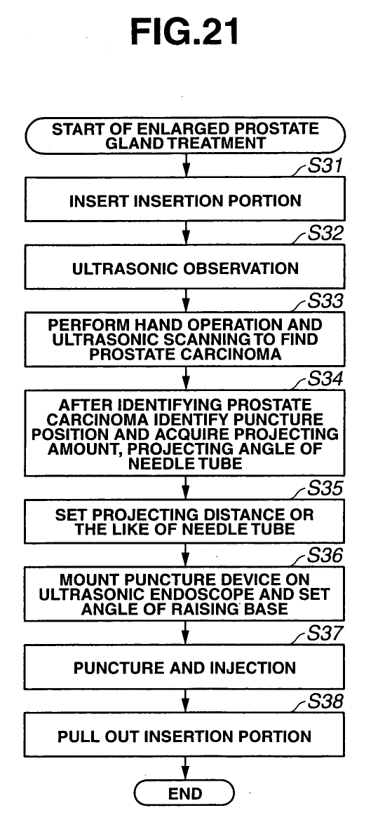

In the transurethral treatment for a prostate carcinoma using the ultrasonic endoscope 2, an operator grasps the size, position or the like of the prostate carcinoma by examining pre-operation images such as CT images and MRI images as necessary. And the ultrasonic endoscope 1 is constituted by preparing the puncture device 6, an RF (Radio Frequency) probe or the like as medical instruments. After that, the operator proceeds with treatment for the prostate carcinoma according to procedures in FIG. 21. In this embodiment, an anticancer agent is filled in a syringe.

First, the operator inserts the insertion portion 11 of the ultrasonic endoscope 2 into the urethra 91 as shown in FIG. 7 as shown in Step S31 in FIG. 21. And the ultrasonic probe 5 provided at the tip end of the insertion portion 11 is inserted toward the prostate gland 92 while performing visual observation by the observing optical system provided at the ultrasonic endoscope 2 and ultrasonic observation by the ultrasonic probe 5 as necessary.

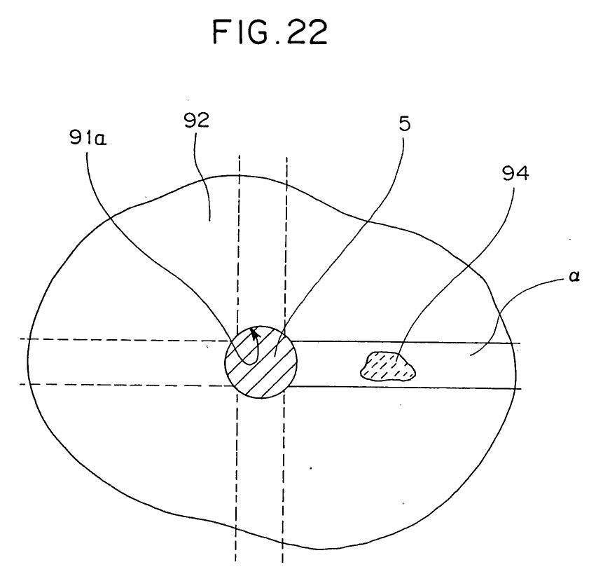

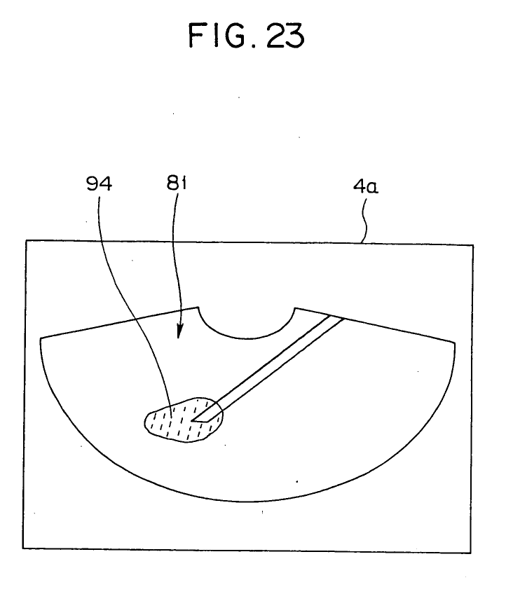

When the ultrasonic probe 5 reaches the vicinity of the prostate gland 92 as shown in FIG. 7, the operator brings the ultrasonic probe 5 into close contact with the urethral wall 91a by curving operation or the like. And as shown in Step S32, the ultrasonic observation is started. Then, as shown in FIG. 8, a B-mode image of the prostate gland 92 is displayed on the screen 4a of the display device 4. Here, the operator observes the B-mode image 81 displayed on the screen 4a of the display device 4 and grasps an observing position by checking a running state of a blood vessel as necessary. After that, the operator carries out work to find a cancer portion in the prostate gland as shown in Step S33. At this time, the operator performs twisting operation of the insertion portion 11 as shown in FIG. 22 or above-mentioned insertion operation, for example, and moves a prostate carcinoma 94 grasped on the pre-operation image to the ultrasonic scanning range α. By this, the prostate carcinoma 94 is displayed on the screen 4a of the display device 4 as shown in FIG. 23. Then the operator moves on to Step S34, checks the prostate carcinoma 94 on the screen and then, acquires the projecting length and projecting angle of the needle tube 61 so that the needle tube 61 is inserted into a desired portion of the prostate carcinoma as shown in FIG. 23.

Next, as shown in Step S35, by manipulating the first fixing screw 8a, the second fixing screw 8b of the puncture device 6, the length of the sheath 62 extending from the tip end of the fixing ring 63 and the projecting length of the needle tube 61 are controlled, and the routine goes on to Step S36. At Step S36, the operator inserts the sheath 62 accommodating the needle tube 61 or the like into the insertion channel 24 from the medical instrument insertion port 19. After that, the fixing ring 63 of the puncture device 6 is connected and fixed to the base portion provided at the medical instrument insertion port 19. By this, the puncture device 6 is mounted at a predetermined position of the ultrasonic endoscope 2 in a predetermined state. After that, the operator sets the tilting angle of the raising base 25 to an angle acquired at Step S34.

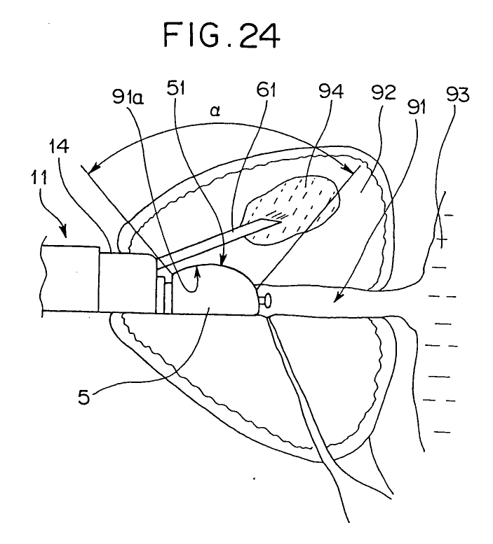

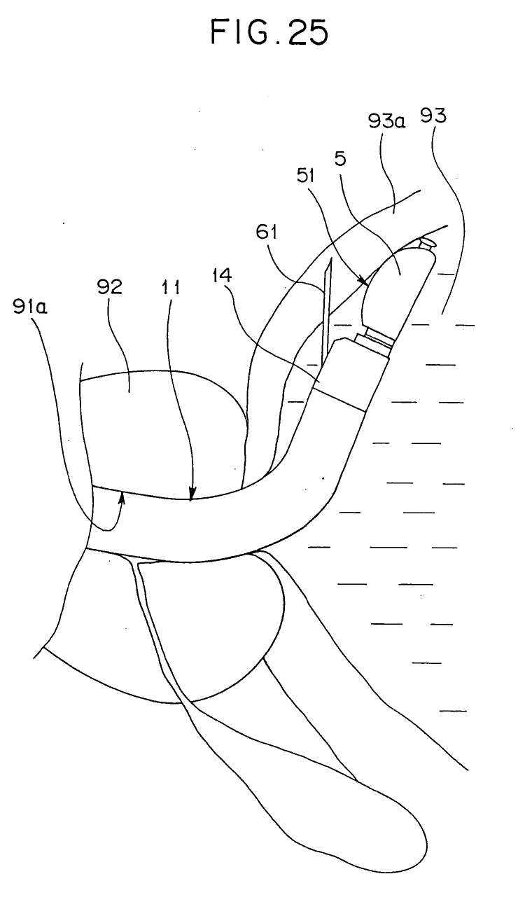

At Step S37, the operator performs puncture by the needle tube 61 toward the prostate carcinoma under ultrasonic guide. Then, as shown in FIG. 24, the needle tube 61 is inserted into the prostate carcinoma. At this time, the operator checks a position of the prostate carcinoma 94, a position of the tip end and a puncture depth or the like of the needle tube 61 on the B-mode image displayed on the screen 4a of the display device 4. When a satisfactory puncture result is obtained, the syringe is attached to the suction base 70, and pushing-in operation is performed to inject a predetermined amount of anticancer agent into the prostate carcinoma 94. After that, the routine goes on to Step S38, where the insertion portion 11 of the ultrasonic endoscope 2 is pulled out of the urethra 91 to finish the treatment. If a satisfactory puncture result is not obtained at Step S37, puncture is performed again.

In this way, the insertion portion of the ultrasonic endoscope is inserted into a urethra and guided to the vicinity of a prostate gland. After that, the needle tube of the puncture device is inserted into a desired position of a prostate carcinoma under ultrasonic observation through the insertion channel provided at the ultrasonic endoscope. After that, an anticancer agent is injected visually to the prostate carcinoma to give transurethral treatment. By this, such treatment is realized that the anticancer agent can be injected accurately after checking a puncture position of the needle tube of the puncture device with respect to the prostate carcinoma on the B-mode image.

In this embodiment, a treatment method of injecting an anticancer agent is shown, but in addition to the treatment method of injecting the anticancer agent, that may be a treatment method using an RF probe by inserting an RF (Radio Frequency) probe into the insertion channel and cauterizing and necrotizing a cancer by an RF electromagnetic wave in combination with an external return electrode or a cryo treatment method of freezing and necrotizing the cancer by inserting a needle-state member for circulating liquid nitrogen through a puncture needle.

The treatment using the RF probe is as described in “Transurethral needle ablation (TUNA): thermal gradient mapping and comparison of lesion size in a tissue model and in patients with benign prostatic hyperplasia” Rasor J S, et al., European Urology, 1993; 24(3): 411-4, PubMed, National Center for Biotechnology Information, National Library of Medicine.

The cryo treatment is as described in “Active rectal wall protection using direct transperineal cryo-needles for histrologically proven prostate adenocarcinomas” Cytron S. et al., European Urology, September 2003; 44(3): 315-20: discussion 320-1, PubMed, National Center for Biotechnology Information, National Library of Medicine.

In the above-mentioned embodiment, a transurethral diagnosis and a treatment for a prostate gland using an ultrasonic endoscope were described, but the diagnosis and treatment may be carried out by inserting the ultrasonic endoscope 2 up to the bladder 93 through the urethra 91 as shown in FIG. 25. In this case, too, the needle tube 61 of the puncture device 6 can be inserted to a desired position in a bladder wall 93a under ultrasonic observation and the treatment can be performed directly by visually checking the position of a bladder cancer (not shown), for example, and the position of the needle tube 61 and injecting an anticancer agent.

Not only administration of the anticancer agent, an effect can be also obtained by the following treatment methods for bladder diseases.

In the case of hyperactive bladder in which contracting action of the bladder 93 occurs frequently, acraturesis or pollakiuria with frequent urge to urinate occurs. As a treatment method for them, injection of botulinus bacillus into a predetermined portion of a bladder wall is known to bring back the contracting action to normal. By using an ultrasonic endoscope, the same treatment as that described for the transhrethral treatment method for an enlarged prostate gland by the ultrasonic endoscope device can be performed.

Moreover, in the case of relaxation of bladder outlet causing acraturesis, there is a treatment method of injecting collagen or the like to the vicinity of the bladder outlet. In this case, too, the depth and position to inject collagen can be identified for treatment under ultrasonic observation.

Furthermore, in the above-mentioned embodiment, a method of diagnosis and treatment using the ultrasonic endoscope 2 is described, but a surgery point can be checked by inserting the ultrasonic endoscope into a urethra when performing an operation.

Specifically, at radical prostatectomy, the ultrasonic probe of the ultrasonic endoscope is arranged in a urethra in the vicinity of a prostate gland. And by the ultrasonic probe, a neurovascular bundle where nerves and vessels are in parallel with each other is displayed on a screen of a display device together with a boundary when the prostate gland is to be separated. Then the neurovascular bundle is displayed on a Doppler image. As a result, at the radical prostatectomy, by inserting the ultrasonic endoscope into the urethra and displaying the neurovascular bundle on the screen of the display device, no cancer tissue is left to be extracted and the neurovascular bundle can be preserved.

And at the radical prostatectomy, a treatment is performed to bind portions where veins are branched to plural at the root. In this treatment, too, the ultrasonic endoscope is inserted into a urethra and the branched veins are displayed on the screen of the display device. By this, the treatment to bind the portion where veins are branched to plural at the root can be performed surely through observation of a Doppler image.

In this way, at operations, by observing the B-mode image or Doppler image obtained by the ultrasonic endoscope inserted into the urethra, the operations can be performed efficiently and safely.

Having described the preferred embodiments of the invention referring to the accompanying drawings, it should be understood that the present invention is not limited to those precise embodiments and various changes and modifications thereof could be made by one skilled in the art without departing from the sprit or scope of the invention as defined in the appended claims.

What is claimed is:

1. A transurethral diagnostic method using an ultrasonic endoscope having an ultrasonic probe, an observation optical system and a channel for inserting a medical instrument comprising steps of:
inserting an insertion portion of the ultrasonic endoscope toward a target diagnostic portion through a urethra;
starting transmission of an ultrasonic wave from the ultrasonic probe which has reached the vicinity of the target diagnostic portion and displaying at least a B-mode image of the target diagnostic portion on a screen;
identifying a puncture position from the B-mode image displayed on the screen; and
puncturing the puncture position identified on the screen by a medical instrument through the channel for inserting the medical instrument.

2. A transurethral treatment method using an ultrasonic endoscope having an ultrasonic probe, an observation optical system and a channel for inserting a medical instrument comprising steps of:
inserting an insertion portion of the ultrasonic endoscope toward a target diagnostic portion through a urethra;
starting transmission of an ultrasonic wave from the ultrasonic probe which has reached the vicinity of the target diagnostic portion and displaying at least a B-mode image of the target diagnostic portion on a screen;
identifying a puncture position from the B-mode image displayed on the screen;
puncturing the target diagnostic portion identified on the screen by a medical instrument through the channel for inserting the medical instrument; and
performing treatment through the medical instrument after checking the puncture position of the medical instrument on the B-mode image displayed on the screen.

3. The transurethral treatment method using an ultrasonic endoscope according to claim 2, wherein, when the treatment step is a treatment step for an enlarged prostate gland, the treatment step includes the step of:
injecting medicinal solution or the like from the medical instrument inserted through the channel for inserting the medical instrument on the B-mode image displayed on the screen.

4. The transurethral treatment method using an ultrasonic endoscope according to claim 2, wherein, when the treatment step is a treatment step for an enlarged prostate gland, the treatment step includes the steps of:
injecting medicinal solution or the like from the medical instrument punctured through the channel for inserting the medical instrument on the B-mode image displayed on the screen;
checking a state of a blood flow on the Doppler image after injecting the medicinal solution from the medical instrument; and
checking a treatment state of the target diagnostic portion from blood flow information displayed on the Doppler image after treatment through the medical instrument.

5. The transurethral treatment method using an ultrasonic endoscope according to claim 3, wherein ethanol is injected in the step of injection.

6. The transurethral treatment method using an ultrasonic endoscope according to claim 4, wherein ethanol is injected in the step of injection.

7. The transurethral treatment method using an ultrasonic endoscope according to claim 3, wherein botulinus bacillus is injected in the step of injection.

8. The transurethral treatment method using an ultrasonic endoscope according to claim 4, wherein botulinus bacillus is injected in the step of injection.

9. The transurethral treatment method using an ultrasonic endoscope according to claim 2, wherein, when the treatment step is a treatment step for a cancer, the treatment step includes the steps of:
displaying a cancer portion on the B-mode image displayed on the screen;
inserting the medical instrument to the cancer portion identified on the screen through the channel for inserting the medical instrument; and
injecting an anticancer agent through the medical instrument after checking the puncture position of the medical instrument on the B-mode image displayed on the screen.

10. The transurethral treatment method using an ultrasonic endoscope according to claim 2, wherein, when the treatment step is a treatment step for a cancer, the treatment step includes the steps of:
displaying a cancer portion on the B-mode image displayed on the screen;
inserting the medical instrument to the cancer portion identified on the screen through the channel for inserting the medical instrument; and
arranging a needle-state member for circulating liquid nitrogen at the cancer portion through the medical instrument so as to freeze and necrotize the cancer, after checking the puncture position of the medical instrument on the B-mode image displayed on the screen.

11. The transurethral treatment method using an ultrasonic endoscope according to claim 2, wherein, when the treatment step is a treatment step for a cancer, the treatment step includes the steps of:
displaying a cancer portion on the B-mode image displayed on the screen;
inserting an RF probe into the cancer portion identified on the screen or arranging the RF probe in the vicinity of the cancer portion through the channel for inserting the medical instrument; and
cauterizing and necrotizing the cancer by an RF electromagnetic wave after checking the position of the medical instrument on the B-mode image displayed on the screen.
